(19) United States
(12) Patent Application Publication
(10) Pub. No.: US 2021/0330348 A1
(43) Pub. Date: Oct. 28, 2021

(54) SYRINGE WITH VISUAL INDICATOR AND RETAINING ELEMENTS

(71) Applicants: Juan Carlos GARCÍA PÉREZ, Gijón (ES); Martin CARPINTERO PORRERO, Gijón (ES)

(72) Inventors: Juan Carlos GARCÍA PÉREZ, Gijón (ES); Martin CARPINTERO PORRERO, Gijón (ES)

(21) Appl. No.: 17/283,954

(22) PCT Filed: Dec. 28, 2018
(86) PCT No.: PCT/ES2018/070841

§ 371 Date: Apr. 9, 2021

(30) Foreign Application Priority Data
Oct. 10, 2018 (ES) U201831541

(51) Int. Cl. A61B 17/34; A61M 5/315; A61M 5/48

(52) U.S. Cl. CPC A61B 17/3401; A61M 5/31505; A61M 2202/048; A61M 2205/583; A61M 2210/1003; A61M 5/486

(57) ABSTRACT

The invention relates to a syringe with a visual indicator and retaining elements, which is used in the application of epidural anaesthesia. The syringe has the particularity of comprising a visual indicator located at the front end of a plunger that drives the fluid contained in the barrel, which visual indicator is an element in the form of a bellows comprising two discs with a membrane between the two discs, wherein the membrane can be deformed according to the pressure in the syringe. The syringe also comprises longitudinal lateral flanges on the body of the plunger, which have a surface with a plurality of teeth, wherein the plunger is rotatable relative to its axial axis, and wherein the teeth allow the plunger to be secured.

### PURPOSE OF THE INVENTION

The present invention relates to a type of medical syringe that is preferably used in epidural anesthesia, which has the particularity of internally comprising two elements, which allow the anesthesiologist to have both a visual reference as to the pressure variation that occurs in the event that the needle is introduced into the epidural cavity, and a retaining element that allows the plunger of the syringe to be locked securing the position thereof and that additionally allows the anesthesiologist to release his/her hands.

The field of the invention relates to medical devices, in particular syringes or syringes, that internally comprise warning or reference means, and also relates to syringes that are particularly used in epidural anesthesia where there is pressure variation between the inside and outside of the epidural cavity.

### BEHIND THE INVENTION

Within the medical field epidural anesthesia or peridural anesthesia is a common and known action, based on the introduction of local anesthetic in the epidural space or cavity, thus blocking the nerve endings at its exit from the spinal cord, and therefore, the nerves that have been reached by said anesthetic are anesthetized.

In this sense, epidural anesthesia requires a medical skill since, like any use of anesthetics, this practice has positive main effects, but applied wrongly it can have negative side effects, for which it is necessary to be able to correctly identify the area to be anesthetized.

For this reason, over the years different types of syringes have been developed that aim to solve this problem of identification of the area. Solutions are highlighted in which warning elements are included in the syringe itself, which allow the anesthesiologist to have a visual reference in case the tip of the needle is not in the correct position.

Disclosure is made in EP2883565 describing a medical syringe intended to identify the epidural space comprising an extendable chamber at the front end of the barrel to which the plunger may be inserted, and just at the junction zone between the barrel and the needle, such that upon a pressure change this chamber expands into the barrel and hinders displacement of the plunger. This solution allows to retain or hinder the movement of the plunger before a pressure change in the epidural cavity, and the chamber actually expands, but it is not a striking visual reference that allows the anesthesiologist to detect such a pressure change in a fast or intuitive way.

In this regard, we also know what is described in U.S. Pat. No. 4,000,741 where a type of medical syringe is taking account, which is applied in an expansive membrane in the barrel pivot, that is, in the junction zone between the barrel and the needle, so that the user of the syringe can be warned of a pressure change. This document allows quick visual reference, since it is located in the needle area, but it is not implementable in epidural anesthesia syringes, where in addition a reference must be made in the plunger area for rapid action by the anesthesiologist.

Document U.S. Pat. No. 5,902,273 is also known, describing a syringe type comprising in a compartment attached to the barrel where a type of extendable membrane is introduced, which modifies its position in the event of a pressure change within the needle barrel. This solution makes it possible to visually detect this pressure change, but requires the development of a special syringe type where the plunger is comprised in a barrel area independent of the area of said membrane, and at the same time the area of connection to the syringe is independent, therefore, the structure of the syringe is complex and beyond the conventional structure of this type of medical instrument.

At this point it is important to define that the fact of being able to detect pressure variation is important, because the epidural space is a virtual cavity located between the two leaves into which the dura mater is divided, where the outermost one of them is confused with the periosteum of the spinal canal, and ends up above in the foramen magnum; while the innermost one is the most properly called dura mater spinalis, and ends in the adult approximately in the second sacral metameter, granting exit there to the terminal end of the marrow, the filum terminalis, and disappearing in the sacrococcygeal ligament itself

In this sense, one of the direct techniques for identifying epidural space is the air resistance loss method, or LOR-A, and the other is LOR-S, loss of resistance-saline; and there are two basic characteristics by which needle entry into the epidural space can be identified: the perceived loss of mechanical strength in the plunger when the needle enters the epidural space and the negative pressure in it.

Regarding the loss of air resistance, it is because, when the tip of the needle is in the interspinous ligament, considerable force can be exerted on the plunger without the air contained in the syringe being expelled from it and, when entering the epidural space, a clear loss of resistance is perceived in the plunger, which allows air to be introduced into said space. In turn, there are at least three modes for performing this technique:
a) as the needle-syringe assembly is continuously advanced, constant pressure is exerted with the thumb of the skilled hand on the plunger;
b) likewise, by continuously advancing the needle-syringe assembly, but exerting intermittent pressure on the plunger by rapid movements, such that air decompression confers a series of rebounds on the plunger;
c) the needle is advanced intermittently, approximately 1 mm each time, testing the pressure resistance exerted by the plunger after each advance.

Regardless of the mode used, once the epidural space has been located, the pressure exerted on the plunger must be interrupted, avoiding any subsequent injection of air, and then an aspiration test can be carried out with which, theoretically, the injected air would be extracted.

As for loss of resistance with liquids, the syringe, instead of air, contains a saline solution or a local anesthetic, and the pressure exerted during needle insertion may be continuous or intermittent. The advantage is that, as the liquids are incomprehensible, the feeling perceived by the anesthesiologist is more fluid.

In any case, the problem can be observed that in order to perform a correct medical practice, the variation between the pressure generated in front of the epidural cavity must be noticed, in order to be sure that the injection has been carried out correctly and that it is in the right area, that is, without puncturing or damaging the dura mater or the second area of the epidural cavity where there could be potentially serious side effects.

Relative to solving this problem is known what is disclosed in document ES2642171, which describes a type of needle for anesthesia tasks, and in particular for use in epidural anesthesia, which allows to improve the LOR-A and LOR-S techniques, which has the peculiarity that at the front end of the plunger that pushes the fluid to be injected into the epidural cavity, there is a piston with an opening that hydraulically connects said part of the internal barrel of the syringe with an internal chamber in the plunger that expands or contracts depending on the pressure, and therefore, in case of such variation, the piston increases its volume and that allows the anesthesiologist to be visually warned that there has been a pressure change, being able at that moment to stop the displacement of the plunger and stopping the injection of anesthetic. This solution, compared to the previous ones, allows to improve the visual warning signal; however, it does not allow the detection and maintenance of the position of the plunger.

In view of the background in the existing background in the state-of-the-art, and the problem of having an element in the same syringe that visually warns of the pressure change and at the same time can stop and retain the displacement of the plunger, the present invention introduces in this sector a solution that combines both functionalities, in such a way that it describes a syringe of normal structure, that is, a transparent barrel that internally comprises a plunger, but that has two elements with which it is achieved:
having a visual reference within the transparent barrel that there has been a pressure variation when the needle is being introduced into the epidural cavity and said area is being anesthetized; for which a plunger is available at the anterior end of, which has a bellows configuration,
and retaining the movement and displacement of the plunger, such that said plunger can be locked in a fixed position, and thus the position of the plunger can be secured and the anesthesiologist can release his/her hands; for which there is a retaining element in the area of the outer reinforcement of the barrel that prevents the plunger from separating from the cylinder, and wherein the plunger has a configuration that allows engagement with said retaining element.

This syringe has the additional advantage over other solutions known and belonging to the state-of-the-art, that its configuration is similar to that of any other syringe, and therefore it is a versatile solution that allows its use in other anesthesia works, not only in epidural anesthesia.

Therefore, by means of the medical syringe described below, the unresolved technical problem of having a syringe that at the same time has a visual pressure change warning element in anesthesia work, and more specifically in epidural anesthesia work, and at the same time has a means of retaining the plunger with which it is ensured that the anesthesia is not applied in an undesired area, allows the position of the plunger to be retained, and additionally allows the anesthesiologist to release his/her right hand so that he/she can introduce the needle-syringe element with two hands together more slowly and more precisely, without having to be aware of the position of the plunger.

### DESCRIPTION OF INVENTION

The invention consists of a medical syringe that is preferably used in epidural anesthesia, having the particularity of an internally-comprised bellows-shaped visual control, which is an element that makes it possible to have a visual reference as to the pressure variation that occurs in the event that the needle is introduced into the epidural cavity, and a retaining element that allows the plunger of the syringe, having a toothed configuration, to be locked and securing the position thereof, and additionally allows the anesthesiologist to release his/her hands.

Going into the detail of the present invention, the syringe essentially comprises the basic components found in any known typology, i.e., comprises a needle; a transparent barrel; a pivot that secures the attachment between the needle and the barrel; a plunger that moves axially within the barrel; an outer plunger support with which the anesthesiologist can move the plunger with his/her hand; and an outer barrel reinforcement that prevents the plunger from separating from the cylinder/barrel. So far it can be seen that the configuration of the syringe is similar to those of any other type of syringe known for other uses; however, the present invention incorporates the two previously mentioned elements with which it is possible to have a reference and visual alert of pressure change, and to be able to lock and adjust the position of the piston, and therefore, providing a solution to the technical problem described above.

The visual indicator that allows visual element is located at the front end of the plunger, and consists of a bellows-shaped structure that it arranges, is configured by two disks, one front and one rear, whose outer parts are flat and in one of them is the front end of the plunger, and which comprise a deformable membrane between them. This membrane is eye-catching in colour, and its compression and/or expansion is carried out when there is a pressure change.

This deformable membrane, with a striking colour when expanded, acts as a visual alert exactly defining the moment when it is placed over the epidural cavity.

Specifically, when the membrane is compressed between the two disks, i.e. hidden, it means that there is pressure and that the needle has not been inserted into the epidural space. However, when the membrane is expanded and visible due to its striking colour, it is because there has been a reduction in pressure, and in particular it means that the needle has been inserted into the epidural space, and therefore, the anesthesiologist has a visual alert that the epidural space has been reached.

On the other hand, the syringe has a retaining element, which consists in that the plunger body comprises at least one longitudinal side flap having a serrated tooth-like configuration, which serves as an anchor and brake on the movement of said plunger. To this end, the outer reinforcement of the barrel that prevents the plunger from separating from the cylinder/barrel has an annular element or retaining ring that serves, after a rotational movement with respect to its axis, to anchor said ring in the teeth of the plunger body. In this manner, by a simple twist of the plunger it is blocked in the ring, and therefore the plunger can be blocked during the needle insertion phase in the patient, which facilitates the anesthesia maneuver since it releases one hand during the anesthesia process.

In a further embodiment of the invention, the barrel has inner hooks that allow the plunger teeth to be fixed thereon, and therefore there is no need for a retaining ring.

The combination of both elements in the same syringe, which additionally has a conventional structure, allows to introduce a versatile solution in this sector, with which all previously defined technical problems are solved.

How to use this syringe to perform epidural anesthesia tasks can be explained from the following steps:
a) in an initial phase where the syringe is outside the patient, the visual indicator is expanded due to lack of pressure. At that moment the filling of air or liquid in the syringe begins, and the piston is released. Once the syringe is loaded, the plunger is rotated to engage the locking teeth.
b) at a later step, the needle is inserted into the patient, but the needle is not yet in the epidural area. As soon as the needle is in the cartilaginous area, the cylinder is pushed, and the visual indicator is compressed and the membrane is hidden due to pressure. The locking system maintains the position of the syringe rod and the pressure, so that it is not necessary to manually maintain the position, and one's hands can be released.
c) finally, the needle reaches the epidural space. Upon reaching this area, there is a significant loss of pressure. The plunger is locked, therefore, it maintains the thrust and pressure on the piston position autonomously, but inside, the core expands and the membrane becomes visible. From this point, the needle can be disengaged and anesthetized. This is possible because the pressure variation in the epidural cavity, where there is minimal pressure, allows the core to expand. In this way it is possible to indicate that the insertion into the epidural space has been correct and that you are in the right area without puncturing, accessing or damaging the dura mater or even the spinal cord, where there could be serious harmful side effects.

In order to complete the description and to help with a better understanding of the features of the invention, a set of figures and drawings is presented in which, by way of illustration and not limitation, the following is represented:

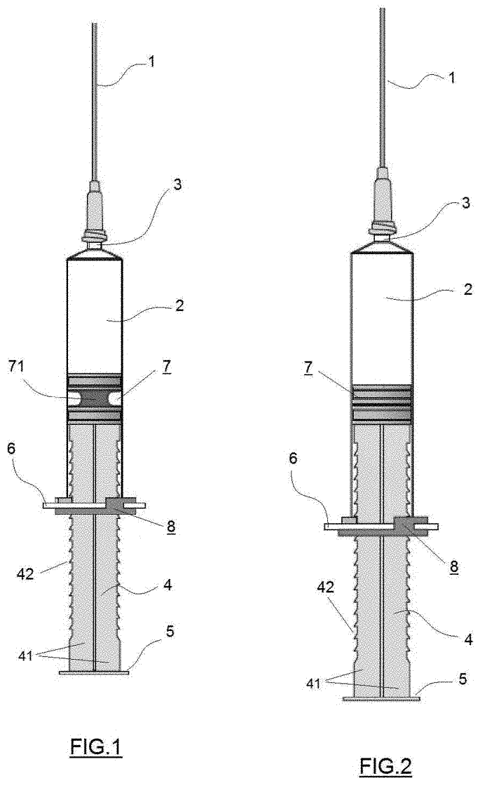

FIG. 1 is a representation of a cross section of the syringe of the invention when the visual indicator is expanded and visible, thus meaning that the needle is within the epidural space, or is outside the patient.

FIG. 2 is a representation of a cross section of the syringe of the invention when the visual control is compressed and hidden, thus meaning that the needle is not within the epidural space.

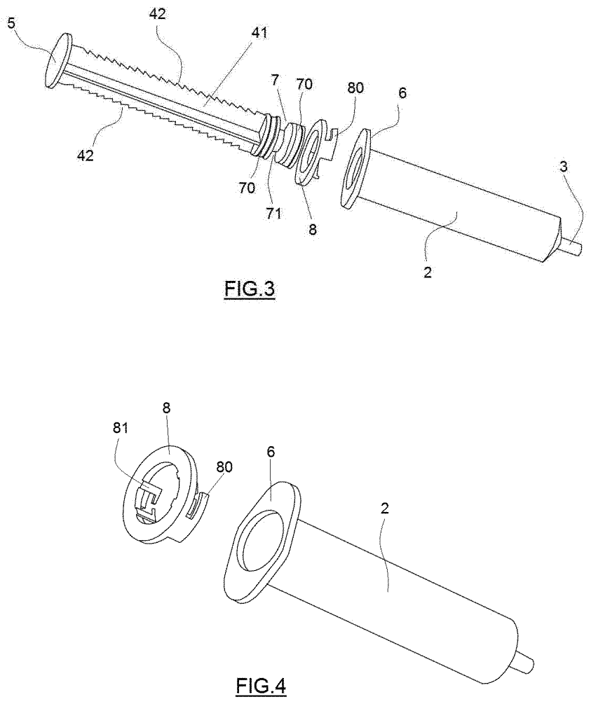

FIG. 3 is an expanded perspective representation of the different elements that make up the syringe, subject of the present invention.

FIG. 4 is a free perspective representation of a detail of the retaining ring.

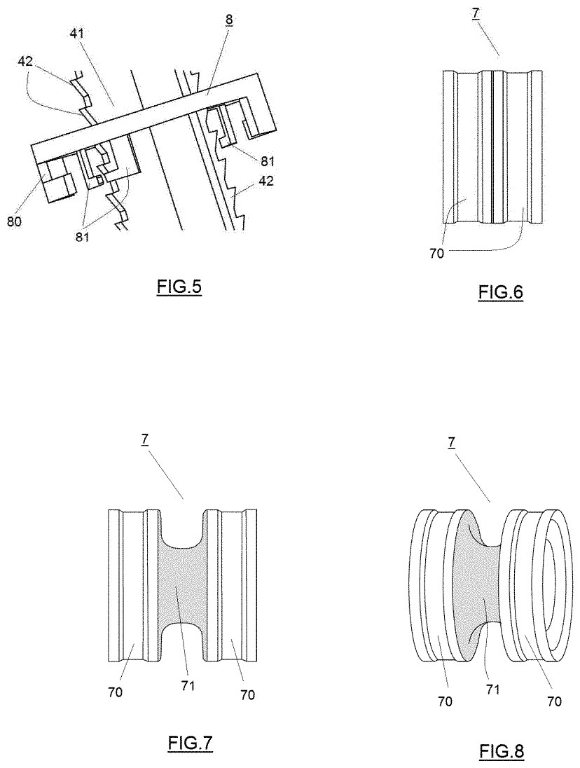

FIG. 5 is a schematic representation of the retention between the ring and the toothed plunger.

FIG. 6 is a detailed representation of the compressed visual indicator.

FIG. 7 is a detailed representation of the expanded visual indicator.

FIG. 8 is a perspective representation, according to the figure above, where the expanded visual indicator can be seen.

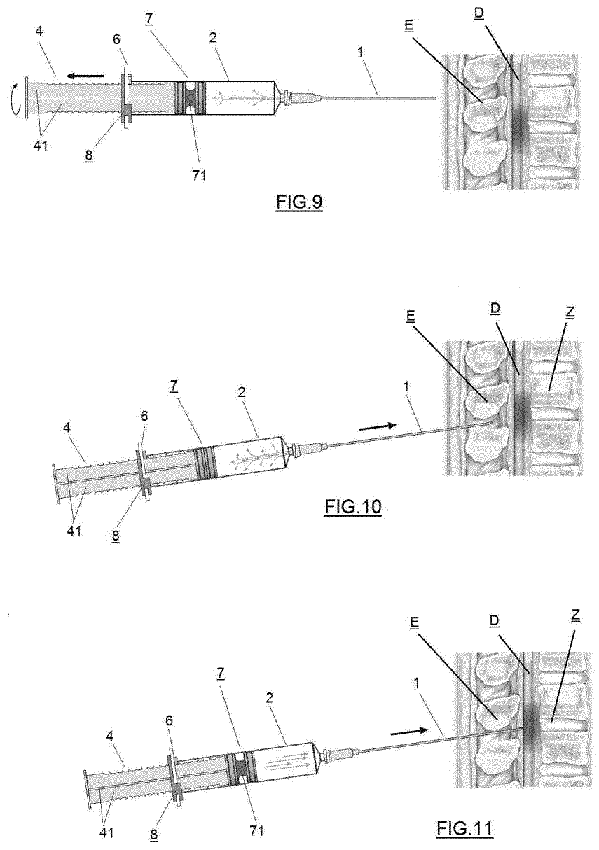

FIG. 9 is a schematic representation of the syringe assembly when outside the patient.

FIG. 10 is a schematic representation of the syringe assembly when inside the patient, but the needle is not in the epidural space.

FIG. 11 is a schematic representation of the syringe assembly when it is within the patient and the needle is within the epidural space.

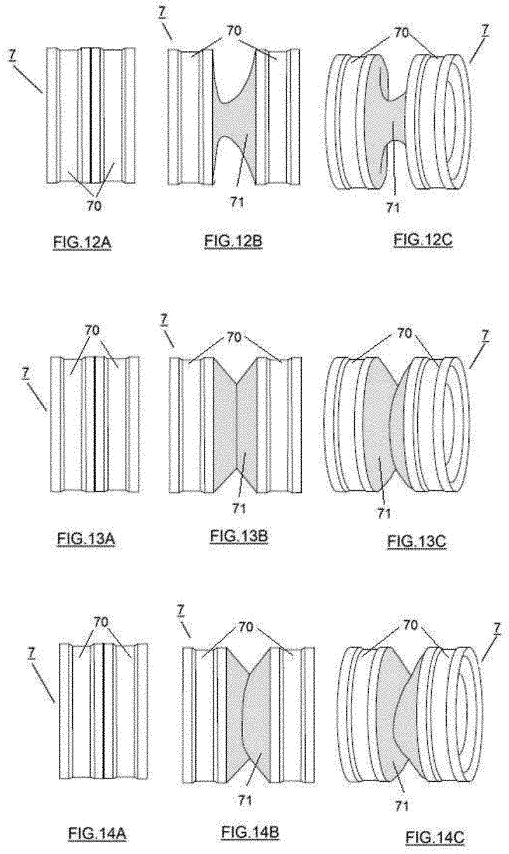

FIGS. 12A to 12C, which are drawings detailing a different type of visual control represented in FIGS. 5 to 7, where the detail of the compressed visual indicator and the expanded visual control is observed.

FIGS. 13A to 13C which are drawings detailing another type of visual control represented in previous figures, where the detail of the compressed visual indicator and the expanded visual indicator is observed.

FIGS. 14A to 14C, which are drawings detailing another type of visual indicator represented in previous figures, where the detail of the compressed visual indicator and the expanded visual indicator is observed.

### DETAILED DESCRIPTION OF THE DRAWINGS OF THE INVENTION

As can be seen in FIGS. 1 to 3, the syringe, subject of the present invention, comprises in a preferred embodiment thereof, the usual elements of this type of medical devices, i.e., comprises a needle (1); a transparent barrel (2); a front pivot (3) that secures the junction between the needle and the barrel; a plunger (4) that moves axially within the barrel; an outer plunger support (5) with which the anesthesiologist can move the plunger with his/her hand; and an outer reinforcement (6) of the barrel that prevents the plunger from separating from the cylinder/barrel.

Additionally, in said figures, and in more detail in FIGS. 4 to 8, in order to solve the technical problem related to having a reference and visual alert of pressure change, and to be able to lock and adjust the position of the plunger, the syringe comprises the following:
a visual indicator (7), which is a bellows-shaped element comprising two disks (70) facing each other and comprising a deformable membrane (71) between them, this membrane being of striking colour, and its compression and/or expansion carried out when there is a pressure change, and acting as a visual alert precisely defining the moment at which it is placed over the epidural cavity (E);
and a restraining element consisting of:
the plunger body (4) has at least one longitudinal side flap (41) having a toothed surface or having a number of teeth (42); and
in the outer reinforcement (6) of the barrel a retaining ring (8) is fitted, which on the one hand has hooks (80) on its outer perimeter that allow it to be fitted and fixed to the outer reinforcement (6), and has inner hooks (81) on its lower face;
so that the teeth (42) can be fitted to the hooks (81) of the retaining ring (8), thus leaving the plunger (4) fixed in its position, and to be released only turn with respect to its axis and release the engagement of said teeth (41) in the retaining ring.

The number of toothed side flaps (41) is variable, it being necessary that there be at least one, and where specifically in FIGS. 1 to 3 one embodiment of the invention can be seen with two flaps. Another aspect is that the faces of the discs (70) of the visual indicator (7) may be flat, as depicted in the Figures, but may have surfaces of another configuration.

Although not depicted, in a second embodiment of the invention the syringe comprises the same elements as usual of any syringe, i.e., a needle (1); a transparent barrel (2) ; a front pivot (3) that secures the junction between the needle and the barrel; a plunger (4) that moves axially within the barrel; an outer plunger support (5) with which the anesthesiologist can move the plunger with his/her hand; and an outer reinforcement (6) of the barrel that prevents the plunger from separating from the cylinder/barrel, but has the particularity that the barrel directly has the inner hooks (81), which are preferably located on the inner face of the outer reinforcement (6) of the barrel, which allow the teeth of the plunger to be fixed thereon, and therefore there is no need for a retaining ring (8).

Additionally, as can be seen in FIGS. 1 and 2, and in greater detail in FIGS. 6 to 8, the syringe may dispose of the visual control (7) in two positions, one in which the membrane (71) is compressed between the two disks (70), i.e., hidden, and means that there is pressure and that the needle has not been introduced into the epidural space; and may also have the expanded visual indicator (7), i.e., that the membrane (71) with its striking colour is visible, which means that there has been a reduction in pressure, and in particular may mean that the needle has been introduced into the epidural space.

In FIGS. 9 to 11 the use of the syringe assembly can be seen schematically, where in FIG. 9 it can be seen that the syringe is outside the patient, and the visual indicator (7) is expanded due to lack of pressure; subsequently, as seen in FIG. 10, but still the needle (1) is not in the epidural zone (E) and the visual indicator (7) is compressed and the membrane is hidden due to pressure; and finally, in FIG. 11, the needle (1) is reached into the epidural space (E), the plunger (4) is blocked and the visual indicator (7) expands and the membrane (71) becomes visible, being able at that time to release the plunger and proceeds to anesthetize. In this way it is possible to indicate that the insertion into the epidural space has been correct and that you are in the right area without puncturing, accessing or damaging the dura mater (D) or the next secondary epidural area (Z), where there could be harmful side effects.

In FIGS. 12A to 14C different possible configurations of the visual indicator (7) can be seen in two positions, one in which the membrane (71) is concealed and means that there is pressure and that the needle has not been introduced into the epidural space (FIGS. 12A, 13A and 14A); and it can also have the expanded visual indicator (7), that is, that the membrane (71) with its striking colour is visible, which means that there has been a reduction in pressure, and in particular it can mean that the needle has been introduced into the epidural space (FIGS. 12B-C, 13B-C and 14B-C).

What is claimed is:

1. A syringe with a visual indicator and retaining elements, for use in the application of epidural anesthesia, which is a syringe comprising a needle (1); a transparent barrel (2) housing the fluid to be applied in the epidural area; a front pivot (3) securing the junction between the needle and the barrel; a plunger (4) moving axially within the barrel; an outer plunger support (5) with which the anesthesiologist can move the plunger with his/her hand; and an outer reinforcement (6) of the barrel, preventing the plunger from separating from the cylinder/barrel; characterized in that the syringe also comprises the following:
a visual indicator (7) located at the front end of the plunger (4), pushing the fluid contained in the barrel (2), which is a bellows-shaped element comprising two disks (70) with a membrane (71) between both disks, wherein the membrane (71) is deformable according to the pressure at which the syringe is located;
at least one longitudinal side flap (41) in the plunger body (4), having a surface with a number of teeth (42), and wherein the plunger (4) is rotatable relative to its axial axis;
inner hooks (81) in the barrel (2); and wherein the teeth (42) of the side fins (41) of the plunger fit into the hooks (81) retaining the position of the plunger (4), and are released by rotating the plunger (4).

2. A syringe with a visual indicator and retaining elements, according to claim 1, characterized in that the retaining ring (8) comprises hooks (80) on its outer perimeter that fit and are fixed in the outer barrel reinforcement (6).

3. A syringe with a visual indicator and retaining elements according to claim 1, characterized in that it comprises a retaining ring (8), which fits into the outer reinforcement (6) of the barrel, and which comprises the inner hooks (81) on its lower face.

4. A syringe with a visual indicator and retaining elements according to claim 1, characterized in that the hooks (81) are located on the inner face of the outer reinforcement (6) of the barrel.

5. A method of use for performing epidural anaesthesia of the syringe with visual indicator according to claim 1, characterized in that it includes the following steps:
a) in an initial phase the visual indicator is expanded and at the beginning of the loading of air or liquid into the syringe, the plunger is rotated to engage the locking teeth.
b) the needle is inserted into the patient and as soon as the needle is in the cartilaginous area, the cylinder is pushed, and the visual indicator is compressed and the membrane is hidden.
c) When the needle reaches the epidural space the indicator expands and the membrane becomes visible.
